(19) United States
(12) Patent Application Publication
(10) Pub. No.: US 2025/0090371 A1
(43) Pub. Date: Mar. 20, 2025

(54) Highly Comfortable Thermal Contact Pad

(71) Applicant: C. R. Bard, Inc., Franklin Lakes, NJ (US)

(72) Inventors: Sean E. Walker, Platteville, CO (US); Madeline Stich, Thornton, CO (US); Gabriel A. Johnston, Broomfield, CO (US); Rebecca D. Salisbury, Lafayette, CO (US); Michael R. Hoglund, Windsor, CO (US)

(21) Appl. No.: 18/728,053

(22) PCT Filed: Jan. 19, 2022
(86) PCT No.: PCT/US2022/013013

§ 371 Date: Jul. 10, 2024

(51) Int. Cl. A61F 7/08; A61F 7/00

(52) U.S. Cl. CPC A61F 7/08; A61F 2007/0054

(57) ABSTRACT

Disclosed herein is a medical pad for exchanging thermal energy between a targeted temperature management (TTM) fluid and a patient. The medical pad includes a fluid containing layer configured for circulation of a TTM fluid therein. The pad is configured to expand together with an expansion of the patient's skin. A fluid containing layer of the pad includes openings extending therethrough to facilitate expansion. The pad further includes tabs extending away from a perimeter edge, so that a clinician may apply a lifting force to the tab to separate a proximate portion of the pad away from the patient. The pad further includes a hydrogel layer formed of an ultraviolet light-cured composition.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled to a patient to affect surface-to-surface thermal energy exchange with the patient. In general, TTM systems include a TTM fluid control module coupled to at least one contact pad via a fluid deliver line. One such system including a thermal contact pad is disclosed in U.S. Published Application No. 2020-0155341 titled “Medical Pad and System for Thermotherpy” filed Oct. 9, 2019, which is incorporated herein by reference in its entirety.

A patient may experience swelling during a TTM therapy which may occur across an area of the skin in contact with the patient. In some instances, the swelling may cause trauma to the skin in contact with the patient, especially along a perimeter edge of the pad. Disclosed here are systems, thermal contact pads, and methods for providing a TTM therapy while minimizing skin trauma.

### SUMMARY OF THE INVENTION

Briefly summarized, disclosed herein is a medical pad for exchanging thermal energy between a targeted temperature management (TTM) fluid and a patient. According to some embodiments, the medical pad includes a fluid containing layer, having a channel structure and a film disposed across an underside of the channel structure, where the film is sealably coupled with the channel structure to form a flow path for the TTM fluid. The pad further includes a plurality of openings extending between a topside of the channel structure and an underside of the film, and a hydrogel layer disposed across the underside of the film, where the hydrogel layer defines a thermal coupling of the fluid containing layer with the patient and an adhesive for adhering the fluid containing layer to a patient's skin. The pad is configured for expansion in at least one direction, so that in use the pad expands together with an expansion of the patient's skin.

In some embodiments, the hydrogel layer comprises an ultraviolet light-cured composition that includes: (i) a cross-linking copolymer in an amount of between about 15% to 30% by weight of the composition, (ii) water in an amount of between about 15% to 40% by weight of the composition, and (iii) glycerol in an amount of between about 25% to 35% by weight of the composition.

In some embodiments, the pad may further comprise one or more tabs coupled with the fluid containing layer, where the tabs extend outwardly away from one or more perimeter edges of the pad. Each tab may be rigidly coupled with the fluid containing layer so that a lifting force applied to the tab causes a separation of a proximate portion of the pad away from the patient. In some embodiments, at least a subset of the tabs are formed of a resilient material and include a portion extending inward from the perimeter edge. In other embodiments, at least a subset of the tabs are formed of an outward extension of the channel structure.

The pad may include an inlet port in fluid communication with a first end of the flow path and an outlet port in fluid communication with a second end of the flow path.

In some embodiments, the expansion of the pad includes an increase of at least one dimension of one or more of the openings.

In some embodiments, the openings include fissures extending inward from a perimeter edge of the pad and across a portion of the pad. In further embodiments, the openings include a first subset of fissures extending inward from a first perimeter edge of the pad and a second subset of fissures extending inward from a second perimeter edge of the pad, where the second perimeter edge is disposed opposite the first perimeter edge. The first subset of fissures and second subset of fissures may be disposed in an alternating arrangement.

In some embodiments, an extendable material is disposed within each fissure. The extendable material is coupled across the fissure from a first side to a second side opposite the first side, and the extendable material is configured to allow a widening of the fissure in accordance with the expansion of the pad. In some embodiments, the extendable material includes a rubber, a woven elasticated material, or a neoprene.

In some embodiments, the channel structure includes a series of interconnected channel segments forming a lattice arrangement, and the openings may include apertures, where each aperture has a circumferential perimeter defined by three or more channel segments. In some embodiments, one or more apertures define one of a rhomboid, square, rectangular, hexagonal, or polygonal shape.

In some embodiments, the pad defines vest configured to extend around a torso of a patient.

Also disclosed herein is a targeted temperature management system that includes a system module configured for preparation and delivery of the TTM fluid and any of the medical pads summarized above fluidly coupled with the system module.

Also disclosed herein is a method of providing a targeted temperature management (TTM) therapy to a patient. According to some embodiments, the method includes (i) applying a thermal contact pad to a patient, where the pad is configured for expansion so that, during the TTM therapy, the pad expands together with an expansion of the patient's skin, (ii) coupling the thermal contact pad with a system module, where the system module is configured for preparation and delivery of a TTM fluid to the thermal contact pad, and (iii) circulating the TTM fluid through a fluid containing layer of the pad to define a thermal energy exchange between the TTM fluid and the patient.

In some embodiments, the method further includes orienting the pad with the patient so that an expansion direction of the pad is aligned with an anticipated expansion direction of the patient's skin.

In some embodiments of the method, the pad further includes one or more tabs coupled with the fluid containing layer, where the tabs extend outwardly away from one or more perimeter edges of the pad, and the method further includes applying a lifting force to one tab of the one or more tabs to lift a proximate portion of the pad away from the patient. The method may further include visually inspecting the area of the skin beneath the proximate portion.

In some embodiments of the method, the pad further comprises a hydrogel layer having an ultraviolet light-cured composition that includes: (i) a cross-linking copolymer in an amount of between about 15% to 30% by weight of said composition, (ii) water in an amount of between about 15% to 40% by weight of said composition, and (iii) glycerol in an amount of between about 25% to 35% by weight of the composition.

In some embodiments of the method, the pad includes a plurality of openings extending between a topside and an underside of the fluid containing layer, and expansion of the pad includes an increase of at least one dimension of one or more of the openings.

In some embodiments of the method, the openings include fissures extending inward from a perimeter edge of the pad and across a portion of the pad.

In some embodiments of the method, the pad further includes an extendable material disposed within each fissure, where the extendable material is coupled across the fissure from a first side to a second side opposite the first side, and the extendable material is configured to allow a widening of the fissure in accordance with the expansion of the pad.

In some embodiments of the method, the fluid containing layer includes a channel structure defining a series of interconnected channel segments forming a lattice arrangement, and the openings include apertures, where each aperture has a circumferential perimeter defined by three or more channel segments.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A illustrates a targeted temperature management (TTM) system including a thermal contact pad coupled with a patient, in accordance with some embodiments;

FIG. 1B illustrates a top view of first embodiment of a thermal contact pad for employment with the system of FIG. 1A, in accordance with some embodiments;

FIG. 1C is a cross-sectional side view of a portion the thermal contact pad of FIG. 1B, in accordance with some embodiments;

FIG. 1D illustrates a top view of the thermal contact pad of FIG. 1B in an expanded state, in accordance with some embodiments;

FIG. 1E is a side view of the thermal contact pad of FIG. 1B applied to the patient, where a portion of the thermal contact pad separated from the patient via a lifting force applied to a tab of the pad, in accordance with some embodiments;

FIG. 2A is an exploded perspective view of a second embodiment of a thermal contact pad, in accordance with some embodiments;

FIG. 2B illustrates a cross-sectional side view of a portion of the thermal contact pad of FIG. 2A, in accordance with some embodiments; and

FIG. 3 is a top view of a third embodiment of a thermal contact pad in the form of a vest, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, fluid, and thermal interaction. Two components may be connected to or coupled with each other even though they are not in direct contact with each other. For example, two components may be coupled with each other through an intermediate component.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Unless defined otherwise, all technical and scientific terms used herein have the same meaning as commonly understood by those of ordinary skill in the art.

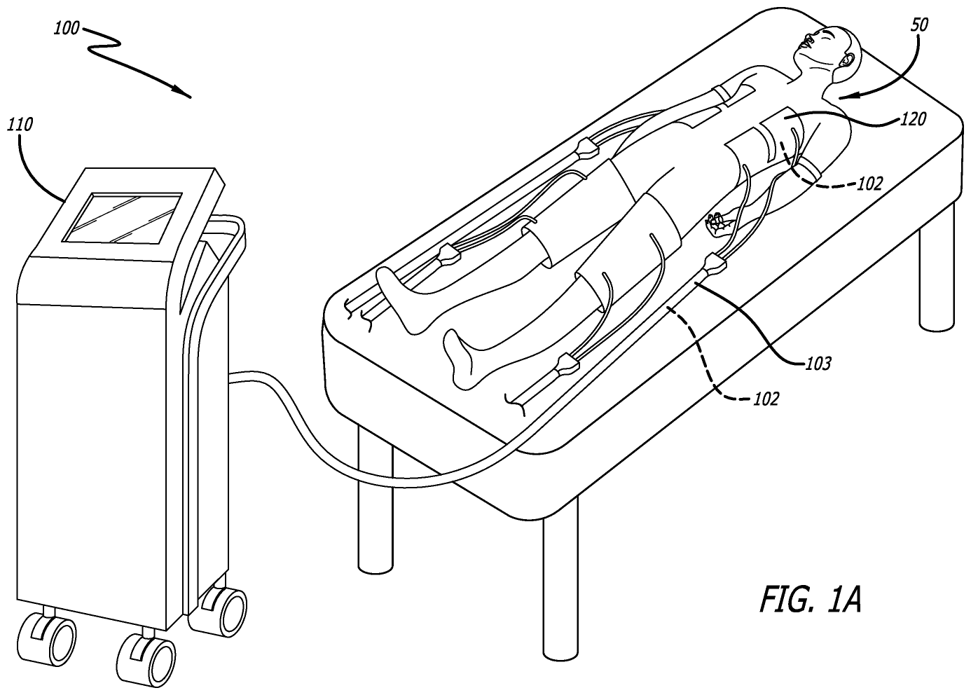

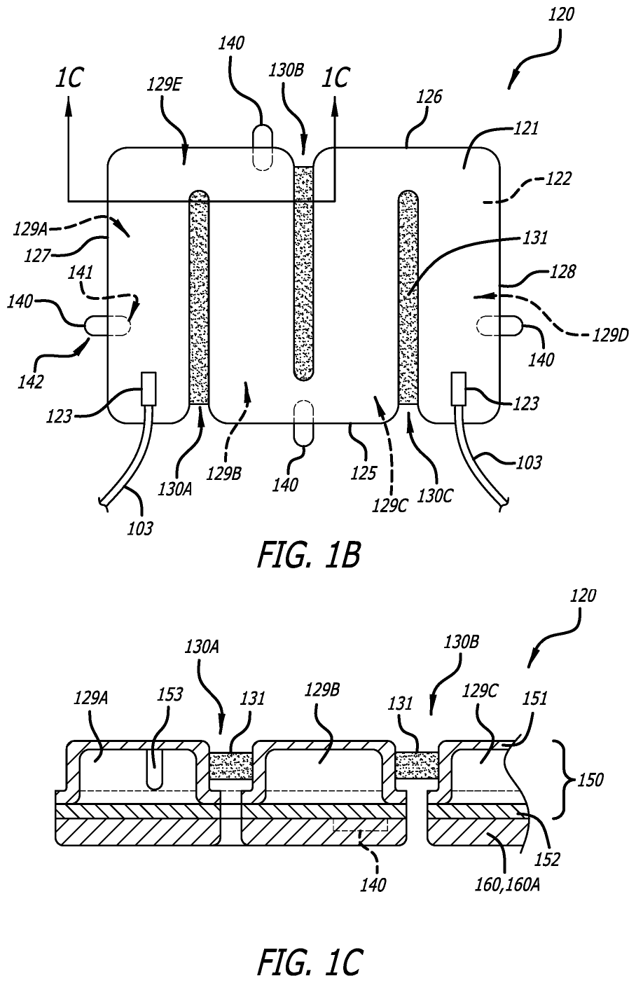

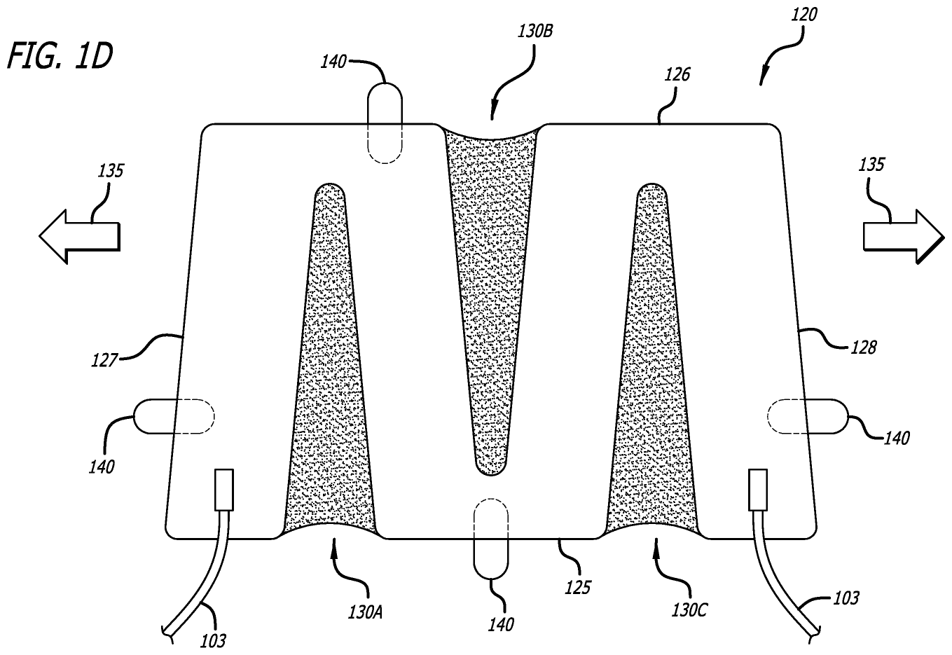

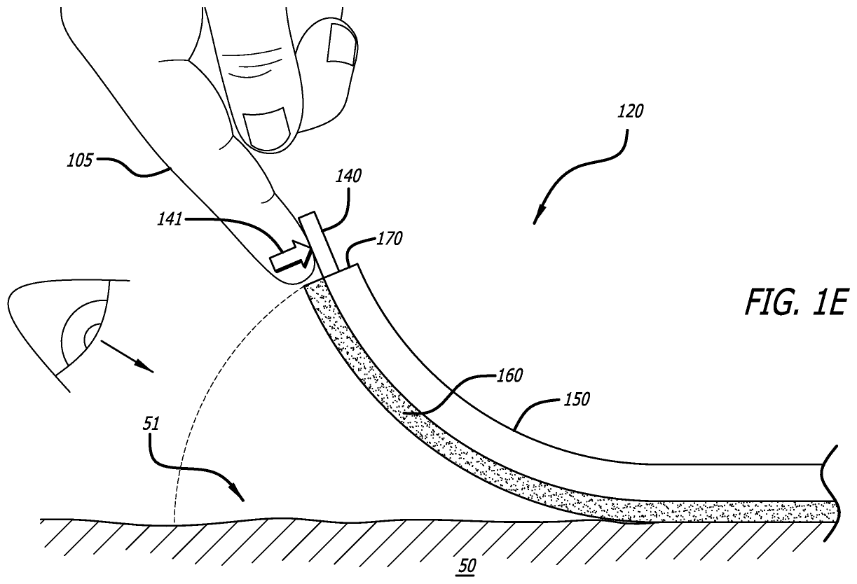

FIG. 1A illustrates a targeted temperature management (TTM) system 100 connected to a patient 50 for administering TTM therapy to the patient 50 which may include a cooling and/or warming of the patient 50, in accordance with some embodiments. The TTM system 100 includes a TTM module 110 for preparing and delivering TTM fluid 102. The TTM system 100 includes a fluid deliver line (FDL) 103 extending from the TTM module 110 to a thermal contact pad (pad) 120 to provide for flow of TTM fluid 102 between the TTM module 110 and the pad 120.

The TTM system 100 may include 1, 2, 3, 4 or more pads 120 and the TTM system 100 may include 1, 2, 3, 4 or more fluid delivery lines 103. In use, the TTM module 110 prepares the TTM fluid 102 for delivery to the pad 120 by heating or cooling the TTM fluid 102 to a defined temperature in accordance with a prescribed TTM therapy. The TTM module 110 circulates the TTM fluid 102 within the pad 120 to facilitate thermal energy exchange with the patient 50. During the TTM therapy, the TTM module 110 may continually control the temperature of the TTM fluid 102 toward a target TTM temperature. As shown, the pad 120 may be applied to different body parts of the patient 50. As such, the pad 120 may be available in different configurations, such as sizes and shapes, for example, to accommodate the different body parts.

The pad 120 may generally define a rectangular shape. In other embodiments, the pad 120 may define shapes other than rectangular such as circular, oval, or a shape that matches or aligns with the shape of a specific body part. In the illustrated embodiment, the pad 120 generally defines a flat shape in a free state, i.e., absent external forces. In other embodiments, the pad 120 may define a curved shape in the free state to accommodate more effectively a non-flat body part, such as a leg for, example.

The pad 120 may be configured to accommodate protrusions and/or depressions along a surface of the patient 50. For example, the pad 120 may be structurally flexible in one or more directions to extend over protrusions and/or fill in depressions of the patient surface so that the pad 120 may define a thermally intimate contact with an uneven skin surface the patient.

The pad 120 may also be configured to change shape along with a changing shape of the patient 50 during the TTM therapy. For example, in some instances, exchanging thermal energy with the patient 50 may cause a portion of the patient to swell. The swelling may cause an area of the skin in contact with the pad 120 to become larger, i.e., stretch in one or more directions. In some embodiments, the pad 120 may be configured to expand in one or more directions together with the stretching of the skin.

FIGS. 1B-1E illustrate a first embodiment of the pad 120 for employment with the targeted temperature management (TTM) system 100, in accordance with some embodiments. FIG. 1B is a top view of the pad 120. The pad 120 generally defines a topside 121 disposed away from the patient 50 during use and an underside 122 configured for contact with the patient 50. For illustration purposes, the pad 120 defines a front side 125, back side 126, a left side 127 and a right side 128. Connectors/ports 123 couple with the FDL 103 to provide for TTM fluid 102 flow into and out of the pad 120. In the illustrated embodiment, the connectors 123 are disposed on the top side 121 of the pad 120. In other embodiments, the connectors may be disposed on the underside 122 or along any of the sides 125-128.

Fissures 130A-130C extend partially across the pad 120. The fissures 130A-130C also extend from the top side 121 to the underside 122 of the pad 120 and a connector material 131 extends across the fissures 130A-130C. The fissures 130A-130C define channels 129A-129D for the flow of TTM fluid 102 therethrough. Flow of TTM fluid 102 within the channels 129A-129D may be unidirectional or bidirectional.

In the illustrated embodiment, the pad 120 includes three fissures 130A-130C. In other embodiments, the pad 120 may include more or fewer than three fissures. The fissures extend inward and partially across the pad 120, i.e., the fissures 130A, 130C extend inward away from the front side 125 toward the back side 126, and the fissure 130B extends inward away from the back side 126 toward the front side 125. In the illustrated embodiment, the three fissures 130A-130C extend inward away from opposites sides of the pad 120 in an alternating arrangement. In other embodiments, the pad 120 may include two or more adjacent fissures extending inward from the same side. Although not shown, in some embodiments, the pad 120 may also include fissures extending inward from the left side 127 and/or the right side 128.

In the illustrated embodiment, the pad 120 includes channel connection portions extending between adjacent channels. For example, a channel connection portion 129E extends between the adjacent channels 129A, 129B to provide for the flow of TTM fluid 102 between the adjacent channels 129A, 129B. Although not shown, in some embodiments, adjacent channels may be fluidly coupled via one or more fluid lines (e.g., tubes) in lieu of a channel connection portion. In such embodiments, a corresponding fissure may extend entirely across the pad 120.

FIG. 1C is a cross-sectional side view of a portion the pad 120, in accordance with some embodiments. The pad 120 includes multiple layers disposed between the topside 105 and the underside 106. The pad 120 generally includes a fluid containing layer 150 having TTM fluid 102 circulating therein, which defines a heat sink or a heat source for the patient 50 in accordance with a temperature of the TTM fluid 102. The fluid containing layer 150 is formed of a channel structure 151 sealably coupled with a film 152 to define channels 129A-129D, i.e., the flow path for the TTM fluid 102 extending between the connectors 123.

The channel structure 151 may be composed of any suitable material such as silicone, a thermoplastic material, for example and may be manufactured via any suitable process, such as thermo-forming, injection molding, or casting, for example. The channel structure 151 may be deflectable so as to form a curve in one or more directions. The deflectability of the channel structure 151 may allow the pad 120 to conform to uneven skin surfaces of the patient 50. The deflectability may allow the pad 120 to extend around or partially around a portion of the patient such as a torso or a leg of the patient, for example.

Although not required, the channel structure 151 may include internal protrusions 153 extending into any or all of the channels 129A-129D toward the film 152 to define sub-channels within the channels 129A-129D. The protrusions 153 may direct the flow of TTM fluid 102 along the channels 129A-129D to inhibit stagnant areas or areas of low flow of the TTM fluid 102. In general, the protrusions 153 may promote an enhanced heat convection between the TTM fluid 102 and the film 152. In some embodiments, the protrusions 153 may extend to and/or be coupled with the film 152. The protrusions 153 may also inhibit collapsing of the channels 129A-129D when a pressure within the channels 129A-129D is negative, i.e., below atmospheric pressure.

The connector material 131 extends across the fissures 130A-130C and couples adjacent channels with each other. For example, the connector material 131 disposed within the fissure 130A extends between the adjacent channels 129A, 129B and couples the channel 129A to the channel 129B. The connector material 131 is a stretchable material. The connector material 131 may be composed of a woven or netting structure to enable the stretchability or the connector material 131 may be stretchable by virtue of its raw material. In some embodiments, the connector material 131 may include a rubber, a woven elasticated material, or a neoprene. In some embodiments, the connector material 131 may be breathable, i.e., provide for the passage of air through the fissure from the top side 121 to the underside 122 of the pad 120. In some embodiments, the connector material 131 may be omitted along all or a portion of a fissure.

With further reference to FIG. 1C, the pad 120 includes a hydrogel layer 160 disposed along the underside 122 of the pad 120. In use the hydrogel layer 160 is in direct contact with the skin of the patient 50 to define a thermally intimate contact between the fluid containing layer 150 and the patient 50. The hydrogel layer 160 may also define an adhesive between the film 152 and the patient's skin. The hydrogel layer 160 may be formed of an ultraviolet light-cured composition 160A that includes (i) a cross-linking copolymer in an amount of between about 15% to 30% by weight of said composition, (ii) water in an amount of between about 15% to 40% by weight of said composition, and (iii) glycerol in an amount of between about 25% to 35% by weight of the composition. In the illustrated embodiment, the hydrogel layer 160 does not extend across the fissures 130A-130C. In other embodiments, the hydrogel layer 160 may extend across the fissures 130A-130C.

FIG. 1D is a top view of the pad 120 in an expanded state/shape. As discussed above the pad 120 is configured to change shape in accordance with changes in shape or size of the patient during the TTM therapy. FIG. 1B illustrates the pad 120 in a non-expanded shape which may, in some embodiments, correlate with a free state of the pad 120, i.e., the shape of the pad 120 absent an external force applied to the pad 120. FIG. 1D illustrates the pad 120 in an exemplary expanded shape.

As shown, a width of the fissure 130B is greater adjacent the back side 126 than proximate the front side 125 so that the length of the pad 120 along the back side 126 is greater in the expanded state than in the non-expanded state. Similarly, the widths of the fissures 130A, 130C are greater adjacent the front side 125 than proximate the back side 126 so that the length of the pad 120 along the front side 125 is greater in the expanded state than in the non-expanded state.

In use, the channels 129A-129D may be adhesively coupled with the skin of the patient via the hydrogel layer 160, such that sliding of the channels 129A-129D with respect to the skin is resisted. The connector material 131 may be sufficiently stretchable to allow widening of the fissure or portion thereof in an instance of patient swelling during the TTM therapy. In other words, the connector material 131 may be sufficiently stretchable to allow the channels 129A-129D move along with the patient's skin during swelling.

With further reference to FIGS. 1B, 1C, the pad 120 includes one or more tabs 140 disposed along any or all of the sides 125-128. The tab 140 includes an internal portion 141 extending inward away from a perimeter edge of the pad 120 and an external portion 142 extend outward away from the perimeter edge. The tab 140 is configured for separating a portion of the pad 120 from the skin along the corresponding perimeter edge proximate the tab 140 when a lifting force is applied to the tab 140. In use, the clinician may utilize the tab 140 to separate the portion of the pad 120 adjacent the tab 140 from the skin to visually inspect the skin adjacent the portion of the pad 120.

The tab(s) 140 may be generally more rigid that the pad 120 so that the clinician may apply a lifting force to the tab 140 to separate a portion of the pad 120 from the patient 50. The resiliency/rigidity of the tab 140 may defined be a material of the tab 140. In the illustrated embodiment, the tab 140 generally defines a thin shape and is composed of rigid material. The tab 140 is coupled with the fluid containing layer 150 at a bottom side of the fluid containing layer 150. In other embodiments, the tab 140 may be coupled with the fluid containing layer 150 along a top side of the fluid containing layer 150 or the top side 121 of the pad 120 at any location between the top side 121 and the underside 122 of the pad 120.

In other embodiments, the tab 140 may be integrally formed with the channeling structure 151. As such, the rigidity of the tab 140 may be defined by a structural shape of the tab 140, e.g., a thickness of the tab 140. In such embodiments, the structure of the channeling structure 151 proximate the tab 140 may define a rigidity of the internal portion 141 of the tab 140. In other words, the tab 140 may be integral to the channeling structure 151, i.e., the tab 140 may be formed of the channeling structure material during the manufacturing process of the channeling structure 151. As such, the tab 140 may be an extension of the channeling structure 151.

FIG. 1E illustrates the pad 120 in use with a patient 50. In some instances, the skin surface adjacent a perimeter edge 170 of the pad 120 may became traumatized as may be caused by frictional contact of the pad 120 with the skin 150, for example. As such, the clinician may desire to inspect the skin 51 for any indications of trauma. Shown is the pad 120 applied to a patient 50. Shown also are the hydrogel layer 160 disposed on an underside of the pad 120 and a tab 140 extending away from the perimeter edge 170. In use, the clinician may apply a lifting force 141 to the tab 140 to lift/separate a portion of the pad 120 away from the patient 50 to inspect a portion of the skin 51 proximate the tab 140 of the pad 120. In lifting the pad 120/220, the clinician may use a finger or thumb 105 to apply the lifting force 141 to the tab 140. By using the tab 140, the clinician is able to avoid touching and disturbing the hydrogel layer 160.

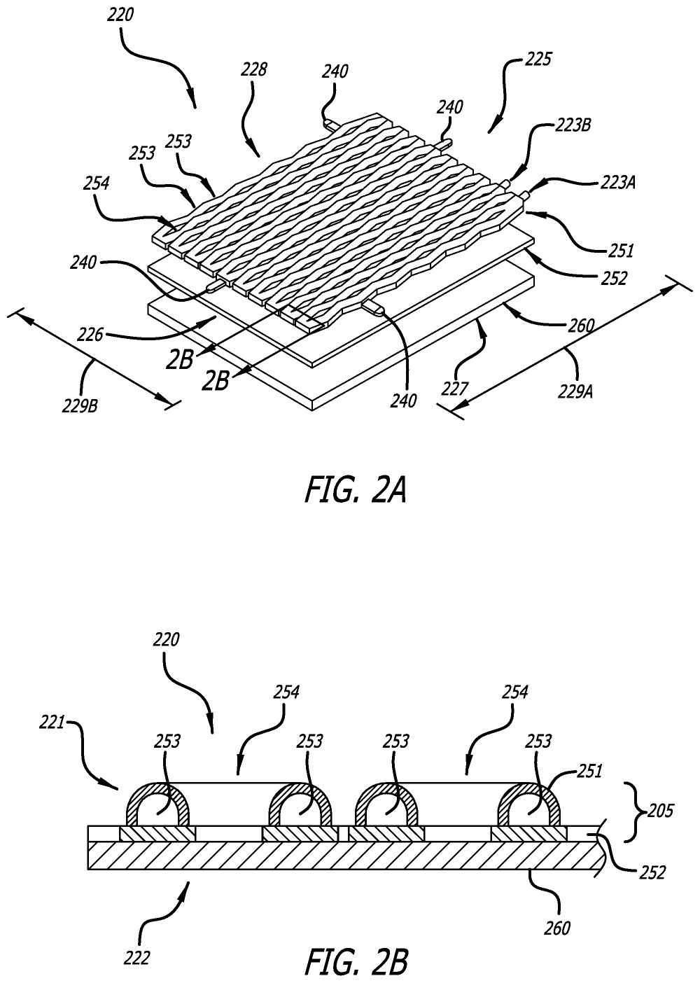

FIGS. 2A-2B is a top view of a second embodiment of a thermal contact pad 220 that can, in certain respects, resemble components of the pad 120 described in connection with FIGS. 1A-1E. It will be appreciated that all the illustrated embodiments may have analogous features. Accordingly, like features are designated with like reference numerals, with the leading digits incremented to “2.” For instance, the fluid containing layer is designated as “150” in FIGS. 1A-1E, and an analogous fluid containing layer is designated “250” in FIGS. 2A-2B. Relevant disclosure set forth above regarding similarly identified features thus may not be repeated hereafter. Moreover, specific features of the thermal contact pad 120 and related components shown in FIGS. 1A-1E may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the thermal contact pad 220 of FIGS. 2A-2B. Any suitable combination of the features, and variations of the same, described with respect to the thermal contact pad 120 and components illustrated in FIGS. 1A-1E can be employed with the thermal contact pad 220 and components of FIGS. 2A-2B, and vice versa. This pattern of disclosure applies equally to further embodiments depicted in subsequent figures and described hereafter.

FIGS. 2A-2B illustrate the thermal contact pad 220 configured for employment with the targeted temperature management (TTM) system 100 (see FIG. 1A), in accordance with some embodiments. FIG. 2A is a perspective exploded view of the thermal contact pad 220 (as viewed from the back left corner) and FIG. 2B is a side cross-sectional view of a portion of the pad 220 in an assembled state cut along sectioning lines 2B-2B. With reference to FIGS. 2A-2B, the pad 220 generally defines a topside 221 disposed away from the patient 50 (see FIG. 1A) during use and an underside 222 configured for contact with the patient 50. For illustration purposes, the pad 220 defines a front side 225, back side 226, a left side 227 and a right side 228.

The fluid containing layer 250 is composed of a channel structure 251 sealably coupled with a film 252 along an underside of the channel structure 251. The channel structure 251 is composed of a series of interconnected channel segments 253 forming, in combination with the film 252, one or more flow paths for TTM fluid 102 extending between an inlet connector 223A and an outlet connector 223B. The interconnected channel segments 253 form a lattice arrangement defining openings 254 that extend through the fluid containing layer 250, i.e., between a top side of the channel structure 251 and a bottom side of the film 252. A shape of the openings 254 may include a diamond, a parallelogram, a rhomboid, a square, a rectangle, a hexagon or any other polygonal shape. Each of the openings 254 may include circumferential perimeter defined by three or more channel segments 253.

The channel structure 251 may be composed of any suitable material such silicone, or a thermoplastic material, for example. The channel structure 251 may be deflectable so as to form a curve in one or more directions. The deflectability of the channel structure 251 may allow the pad 220 to conform to uneven skin surfaces of the patient 50. The deflectability may allow the pad 220 to extend around or partially around a portion of the patient such as a torso or leg of the patient, for example.

The pad 220 may generally define a rectangular shape defining a length 229A extending between the front side 225 and the back side 226 and a width 229B extending between the left side 227 and the right side 228. In a free state, the length 229A may be longer than the width 229B for vice versa.

The channel structure 251 is configured to change shape in response to an external force. For example, a tension force between the left and right sides 227, 228 may cause an increase in the width 229B. Similarly, a tension force between the front and back sides 225, 226 may cause an increase in the length 229A. In some embodiments, an increase in length may result in a decrease in width and vice versa. In some embodiments, the pad 120 may more easily increase in width than in length, i.e., require a lower tension force to cause an expansion.

The pad 220 includes a hydrogel layer 260 disposed along the underside of the pad 220. In use the hydrogel layer 260 is in direct contact with the skin of the patient 50 to define a thermally intimate contact between the fluid containing layer 250 and the patient 50. The hydrogel layer 260 may be composed of a material similar to the ultraviolet light-cured composition 160A of FIG. 1C. In the illustrated embodiment, the hydrogel layer 260 may extend across the openings 253. In some embodiments, one or more openings 253 may extend through the hydrogel layer 260.

The pad 220 may include one or more tabs 240 disposed along any or all of the sides 225-228. The tab 240 extend away from the perimeter edge of the pad 220. The tab(s) 140 may be integrally formed with the channeling structure 251. As such, the channeling structure 251 may help define a rigidity of the tab 240. The tab(s) 240 may be generally more rigid that the pad 220 so that the clinician may apply a lifting force to the tab 240 to separate a portion of the pad 220 from the patient 50. In some embodiments, the tab 240 may be a separate component coupled with the fluid containing layer 250. The rigidity of the tab 240 may be defined by a raw material of the tab 240.

Although not shown, the thermal contact pad 120 of FIGS. 1A-1E and the thermal contact pad 220 of FIGS. 2A-2B may include a combination of the channel structure 151 and the channel structure 251.

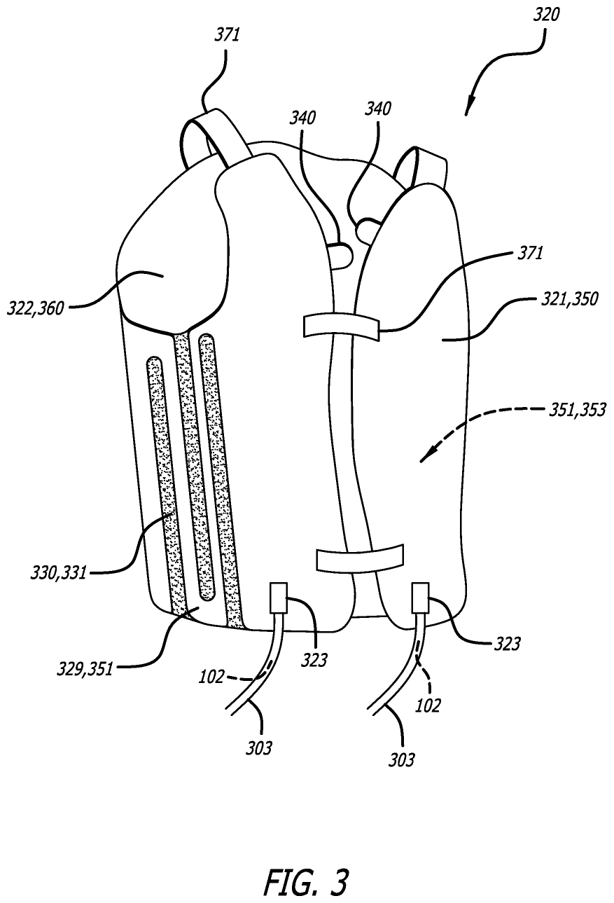

FIG. 3 illustrates a third embodiment of the pad 320 in the form of a vest configured to extend around a torso of the patient 50 (see FIG. 1A). The pad 320 defines a topside/outerside 321 and an underside 322. Disposed along the underside 322 is the hydrogel layer 360. Connectors 323 couple the FDL 303 to the pad 320 to provide for the flow of TTM fluid 102 to the pad 320. The pad 320 includes a plurality of fissures 330 to provide for expandable portions of the pad 320. As illustrated, one expandable portion may be disposed along a right side (e.g., adjacent the right arm of the patient) of the pad 320. Although hidden in FIG. 3, the pad 320 may include another expandable portion disposed along the left side opposite the right side. In some embodiments, the pad 320 may include additional expandable portions. For example, in some embodiments, the pad 320 may include expandable portions disposed along a front side and/or a back side of the pad 320.

The pad 320 includes a fluid containing layer 350 disposed along the outerside 321. The channels 329 disposed between the fissures 330 provide for the flow of the TTM fluid across the expandable portions. The fluid containing layer 350 includes a channel structure 351 extending along the expandable portions and along non-expandable portions. Although not required, the channel structure 351 may include internal protrusions 353. The protrusions 353 may define sub-channels within the fluid containing layer 350 direct the flow of TTM fluid 102 so as to inhibit stagnant areas or areas of low flow of the TTM fluid 102. In general, the protrusions 353 may promote an enhanced thermal energy exchange between the TTM fluid 102 and the patient 50. The protrusions 353 may also inhibit collapsing fluid containing layer 350 when a pressure within the fluid containing layer 350 is negative, i.e., below atmospheric pressure.

The pad 320 may include fastening devices 371 to provide for the attachment of adjacent portions of the pad 320. For example, a pair of fastening devices 371 may extend between the front side and the back side of the pad 320 so as to extend over a shoulder of the patient 50. Other fastening devices 371 may extend between right and left front portions of the pad 320. The fastening devices 371 may provide for selective attachment and detachment of the adjacent portions of the pad 320.

The pad 320 includes one or more tabs 340 disposed on perimeter edges of the pad 320 to provide for skin inspections as shown and described above (see FIG. 1E).

Although not shown, in some embodiments, the pad 320 (or more specifically the fluid containing layer 350) may include lattice channel structure (see FIGS. 2A-2B) in leu of or in addition to the fissures 330. For example, one or more portions of the front side and/or back side may include a lattice channeling structure.

A method of providing a targeted temperature management (TTM) therapy to a patient may include all or a subset of the following steps or processes. A clinician may apply the thermal contact pad to a patient over a defined area of the patient to facilitate thermal energy exchange with the patient. The clinician may couple the pad with a system module, and initiate a circulation the TTM fluid through the fluid containing layer of the pad to commence the thermal energy exchange between the TTM fluid and the patient.

As the pad may be configured to expand in a single direction (or expand more readily in a single direction), the clinician may orient the pad to align with an anticipated direction of expansion of the patient's skin. For example, in some instances, a thigh of the patient may expand/swell more significantly along a circumference of the thigh rather than along the length of the thigh. As such, the clinician may orient the pad to align the direction of pad expansion with the circumference of the thigh.

In performing the method, the clinician may apply a lifting force to a tab of the pad to lift/separate a portion of the pad proximate the tab away from the patient so as to expose the skin the proximate portion of the pad. The clinician may then visually inspect the skin beneath the proximate portion to check for potential skin trauma. The clinician may engage the tab to avoid touching the hydrogel layer.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims.

1. A medical pad for exchanging thermal energy between a targeted temperature management (TTM) fluid and a patient, the pad comprising
a fluid containing layer, comprising:
a channel structure; and
a film disposed across an underside of the channel structure, the film sealably coupled with the channel structure to form a flow path for the TTM fluid;
a plurality of openings extending between a topside of the channel structure and an underside of the film; and
a hydrogel layer disposed across the underside of the film, the hydrogel layer defining:
a thermal coupling of the fluid containing layer with the patient, and
an adhesive for adhering the fluid containing layer to a patient's skin,
wherein the pad is configured to increase in length in at least one direction so that, in use, the pad increases in length together with an increase in length of the patient's skin.

2. The medical pad of claim 1, wherein the hydrogel layer comprises an ultraviolet light-cured composition that includes:
a cross-linking copolymer in an amount of between about 15% to 30% by weight of the composition,
water in an amount of between about 15% to 40% by weight of the composition, and
glycerol in an amount of between about 25% to 35% by weight of the composition.

3. The medical pad of claim 1, further comprising:
one or more tabs coupled with the fluid containing layer, the tabs extending outwardly away from one or more perimeter edges of the pad.

4. The medical pad of claim 3, wherein each tab is coupled with the fluid containing layer so that a lifting force applied to the tab causes a separation of a proximate portion of the pad away from the patient.

5. The medical pad of claim 3, wherein at least a subset of the tabs:
are formed of a resilient material, and
include a portion extending inward from the perimeter edge.

6. The medical pad of claim 3, wherein at least a subset of the tabs are formed of an outward extension of the channel structure.

7. The medical pad of claim 1, further comprising:
an inlet port in fluid communication with a first end of the flow path; and
an outlet port in fluid communication with a second end of the flow path.

8. The medical pad of claim 1, wherein the expansion of the pad includes an increase of at least one dimension of one or more of the openings.

9. The medical pad of claim 8, wherein the openings include fissures extending:
inward from a perimeter edge of the pad, and
across a portion of the pad.

10. The medical pad of claim 9, wherein the openings include:
a first subset of fissures extending inward from a first perimeter edge of the pad, and
a second subset of fissures extending inward from a second perimeter edge of the pad, the second perimeter edge opposite the first perimeter edge.

11. The medical pad of claim 10, wherein the first subset of fissures and second subset of fissures are disposed in an alternating arrangement.

12. The medical pad of claim 9, further comprising an extendable material disposed within each fissure, wherein the extendable material:
is coupled across the fissure from a first side to a second side opposite the first side, and
is configured to allow a widening of the fissure in accordance with the expansion of the pad.

13. The medical pad of claim 12, wherein the extendable material includes a rubber, a woven elasticated material, or a neoprene.

14. The medical pad of claim 1, wherein the channel structure includes a series of interconnected channel segments forming a lattice arrangement.

15. The medical pad of claim 14, wherein the openings include apertures, each aperture having a circumferential perimeter defined by three or more channel segments.

16. The medical pad of claim 15, wherein one or more apertures define one of a rhomboid, square, rectangular, hexagonal, or polygonal shape.

17. The medical pad of claim 1, wherein the pad defines a vest configured to extend around a torso of a patient.

18. A targeted temperature management system, comprising:
a system module configured for preparation and delivery of the TTM fluid; and
the medical pad of claim 1 fluidly coupled with the system module.

19-27. (canceled)
